(19) United States
(12) Patent Application Publication
(10) Pub. No.: US 2012/0059190 A1
(43) Pub. Date: Mar. 8, 2012

(54) NOVEL NITRATION OF TETRACYCLINES

(75) Inventors: ChunHua Yang, Edison, NJ (US); John Leo Dillon, JR., Tinton Falls, NJ (US); Ramachandra Naik, Maharashtra (IN)

(73) Assignee: Wyeth LLC, Madison, NJ (US)

(21) Appl. No.: 13/255,642

(22) PCT Filed: Mar. 9, 2010
(86) PCT No.: PCT/US10/26630

§ 371 Date: Nov. 22, 2011

Related U.S. Application Data
(60) Provisional application No. 61/159,466, filed on Mar. 12, 2009.

(51) Int. Cl. C07C 231/12; C07C 231/08

(52) U.S. Cl. 564/146; 564/157

(57) ABSTRACT

The invention in one embodiment is directed to a method of preparing a compound of formula 1,

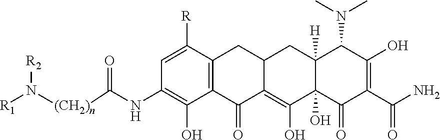

or a pharmaceutically acceptable salt thereof, wherein R₁ and R₂ are each independently chosen from hydrogen, (C₁-C₆)alkyl, and cycloalkyl, R is —NR₃R₄, where R₃ and R₄ are each independently chosen from hydrogen, and (C₁-C₄)alkyl; and n ranges from 1-4, comprising:

(a) reacting a C₁-C₁₂ alkyl nitrate with a compound of formula 2,

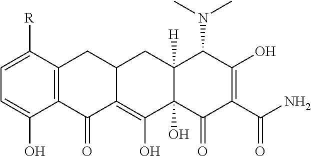

or a salt thereof, in the presence of an acid at a concentration greater than 70% weight of acid/weight of solution, the acid being selected from the group consisting of sulfuric acid, and R₅—SO₃H wherein R₅ is C₁-C₄ alkyl optionally substituted with one or more halogen, or R₅ is C₆-C₁₀ aryl optionally substituted with one or more C₁-C₄ alkyl or halogen, to produce a reaction mixture containing a compound of formula 3 or a salt thereof;

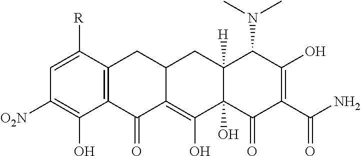

(b) reducing the compound of formula 3 or a salt thereof to form a compound of formula 4 or a salt thereof

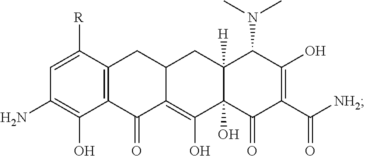

(c) acylating the compound of formula 4 to form a compound of formula 1; and

(d) optionally forming a pharmaceutically acceptable salt of the compound of formula 1.

### CROSS-REFERENCE TO RELATED APPLICATIONS

This application is the national stage filing under 35 U.S.C. 371, of Patent Cooperation Treaty (PCT) Patent Application No. PCT/US2010/026630, filed on Mar. 9, 2010, which claims the benefit of U.S. Provisional Application No. 61/159,466, filed Mar. 12, 2009, the disclosures of which are incorporated herein by reference in their entirety.

### BACKGROUND OF THE INVENTION

Tigecycline was developed in response to the worldwide threat of emerging resistance to antibiotics. Tigecycline has expanded broad-spectrum antibacterial activity both in vitro and in vivo. Glycylcycline antibiotics, like tetracycline antibiotics, act by inhibiting protein translation in bacteria.

Tigecycline is a known antibiotic in the tetracycline family and a chemical analog of minocycline. It may be used as a treatment against drug-resistant bacteria, and it has been shown to work where other antibiotics have failed. Tigecycline may be used in the treatment of many bacterial infections, such as complicated intra-abdominal infections (cIAI), complicated skin and skin structure infections (cSSSI), Community Acquired Pneumonia (CAP), and Hospital Acquired Pneumonia (HAP) indications, which may be caused by gram-negative and gram-positive pathogens, anaerobes, and both methicillin-susceptible and methicillin-resistant strains of Staphylococcus aureus (MSSA and MRSA). Additionally, tigecycline may be used to treat or control bacterial infections in warm-blooded animals caused by bacteria having the TetM and TetK resistant determinants. Also, tigecycline may be used to treat bone and joint infections, catheter-related Neutropenia, obstetrics and gynecological infections, or to treat other resistant pathogens, such as VRE, ESBL, enterics, rapid growing mycobacteria, and the like.

Tigecycline suffers some disadvantages in that it may degrade by epimerization. Epimerization is a known degradation pathway in tetracyclines generally, although the rate of degradation may vary depending upon the tetracycline. Comparatively, the epimerization rate of tigecycline may be fast, even for example, under mildly acidic conditions and/or at mildly elevated temperatures. The tetracycline literature reports several methods scientists have used to try and minimize epimer formation in tetracyclines. In some methods, the formation of calcium, magnesium, zinc or aluminum metal salts with tetracyclines limit epimer formation when done at basic pHs in non-aqueous solutions. (Gordon, P. N, Stephens Jr, C. R., Noseworthy, M. M., Teare, F. W., U.K. Patent No. 901,107). In other methods, (Tobkes, U.S. Pat. No. 4,038,315) the formation of a metal complex is performed at acidic pH and a stable solid form of the drug is subsequently prepared.

Tigecycline differs structurally from its epimer in only one respect. In tigecycline, the N-dimethyl group at the 4 carbon is cis to the adjacent hydrogen as shown in formula I

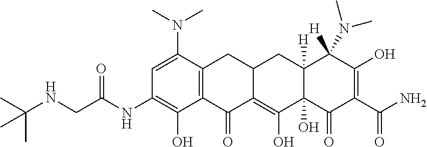

whereas in the epimer (i.e., the C₄-epimer) they are trans to one another in the manner indicated in the structure below:

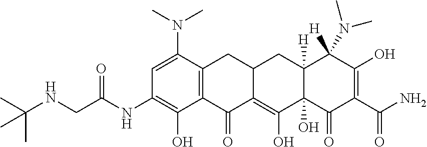

Although the tigecycline epimer is believed to be non-toxic, under certain conditions it may lack the anti-bacterial efficacy of tigecycline and may, therefore, be an undesirable degradation product. Moreover, the amount of epimerization can be magnified when synthesizing tigecycline in a large scale.

Other methods for reducing epimer formation include maintaining pHs of greater than about 6.0 during processing; avoiding contact with conjugates of weak acids such as formates, acetates, phosphates, or boronates; and avoiding contact with moisture including water-based solutions. With regard to moisture protection, Noseworthy and Spiegel (U.S. Pat. No. 3,026,248) and Nash and Haeger, (U.S. Pat. No. 3,219,529) have proposed formulating tetracycline analogs in non-aqueous vehicles to improve drug stability. However, most of the vehicles included in these disclosures are more appropriate for topical than parenteral use. Tetracycline epimerization is also known to be temperature dependent so production and storage of tetracyclines at low temperatures can also reduce the rate of epimer formation (Yuen, P. H., Sokoloski, T. D., J. Pharm. Sci. 66:1648-1650, 1977; Pawelczyk, E., Matlak, B, Pol. J. Pharmacol. Pharm. 34: 409-421, 1982). Several of these methods have been attempted with tigecycline but apparently none have succeeded in reducing both epimer formation and oxidative degradation while not introducing additional degradants. Metal complexation, for example, was found to have little affect on either epimer formation or degradation generally at basic pH.

Although the use of phosphate, acetate, and citrate buffers improve solution state stability, they seem to accelerate degradation of tigecycline in the lyophilized state. Even without a buffer, however, epimerization is a more serious problem with tigecycline than with other tetracyclines such as minocycline.

In addition to the C₄-epimer, other impurities include oxidation by-products. Some of these by-products are obtained by oxidation of the D ring of the molecule, which is an aminophenol. Compounds of formula 3 (see Scheme 1 herein) can be readily oxidized at the C-11 and C-12a positions. Isolation of compounds of formula 3 by precipitation with a non-solvent can have the problem that oxidation by-products and metal salts coprecipitate with the product resulting in very low purities. The oxidation and degradation of the nucleus of compounds of formula 3 can be more pronounced under basic reaction conditions and more so on large-scale operations since processing times are typically longer and the compounds are in contact with the base for a longer time.

Moreover, degradation products may be obtained during each of the different synthetic steps of a scheme, and separating the required compound from these degradation products can be tedious. For example, conventional purification techniques, such as chromatography on silica gel or preparative HPLC cannot be used to purify these compounds easily because of their chelating properties. Although some tetracyclines have been purified by partition chromatography using columns made of diatomaceous earth impregnated with buffered stationary phases containing sequestering agents like EDTA, these techniques can suffer from very low resolution, reproducibility and capacity. These disadvantages may hamper a large-scale synthesis. HPLC has also been used for purification, but adequate resolution of the various components on the HPLC columns requires the presence of ion-pairing agents in the mobile phase. Separating the final product from the sequestering and ion-pairing agents in the mobile phase can be difficult.

While on a small-scale the impure compounds obtained by precipitation may be purified by preparative reverse-phase HPLC, purification by reverse phase liquid chromatography can be inefficient and expensive when dealing with kilogram quantities of material.

Processes for the formation of compounds such as tigecycline, a compound of formula 1, or structurally related compounds have been described in U.S. Patent Publication Nos. 2007-0026080A1, 2007-0049560A1, 2007-0049563A1, 2007-0049562A1, 2007-0049561A1, and in U.S. patent application Ser. No. 12/251,488, each of which is incorporated by reference herein in its entirety. However, there remains a need to obtain the one compound of formula 1 in a more purified form than previously achieved. There also remains a need for new syntheses to minimize use of chromatography for purification.

In the preparation of tigecycline disclosed in U.S. Patent Publication No. 2007-0049560, as an example, nitration of minocycline with nitric acid is one of the steps of the process. Nitration of tetracyclines with nitric acid has also been described in U.S. 2007-0244335. Nitration of other chemical compounds with alkyl nitrates has been reported in WO 2003/011810, U.S. Pat. No. 3,694,513, Chem. Rev. 1955, 55, 485-510, J. Am. Chem. Soc. 1974, 96, 2892-98, U.S. Pat. No. 2,4169,74, and U.S. Pat. No. 7,005,553. However, the use of alkyl nitrates for the nitration of minocycline has not been reported.

### SUMMARY OF THE INVENTION

In one embodiment the invention is directed to a method of preparing a compound of formula 1,

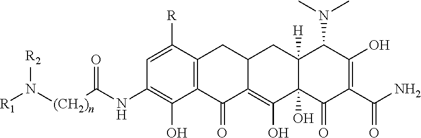

or a pharmaceutically acceptable salt thereof, wherein R₁ and R₂ are each independently chosen from hydrogen, (C₁-C₆)alkyl, and cycloalkyl,; R is —NR₃R₄, where R₃ and R₄ are each independently chosen from hydrogen, and (C₁-C₄)alkyl; and n ranges from 1-4, comprising:

(a) reacting a C₁-C₁₂ alkyl nitrate with a compound of formula 2,

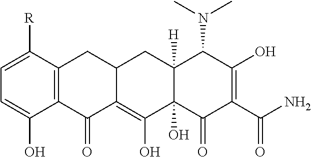

or a salt thereof, in the presence of an acid at a concentration greater than 70% weight of acid/weight of solution,the acid being selected from the group consisting of sulfuric acid and R₅—SO₃H wherein R₅ is C₁-C₄ alkyl optionally substituted with one or more halogen, or R₅ is C₆-C₁₀ aryl optionally substituted with one or more C₁-C₄ alkyl or halogen, to produce a reaction mixture containing a compound of formula 3 or a salt thereof

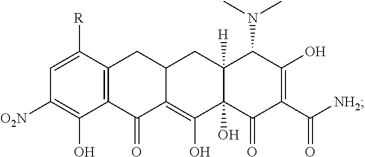

(b) reducing the compound of formula 3 or a salt thereof to form a compound of formula 4 or a salt thereof

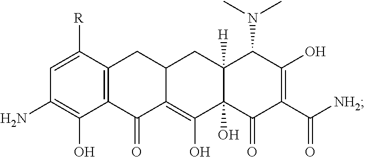

(c) acylating the compound of formula 4 to form a compound of formula 1; and

(d) optionally forming a pharmaceutically acceptable salt of the compound of formula 1.

The invention in another embodiment is directed to a method of preparing a compound of formula 3,

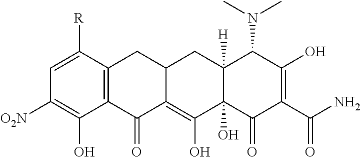

or a salt thereof, wherein R₁ and R₂ are each independently chosen from hydrogen, (C₁-C₆)alkyl, and cycloalkyl; R is —NR₃R₄, where R₃ and R₄ are each independently chosen from hydrogen, and (C₁-C₄)alkyl; and n ranges from 1-4, comprising reacting a C₁-C₁₂ alkyl nitrate with a compound of formula 2,

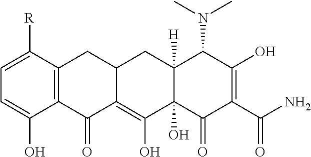

or a salt thereof, in the presence of an acid at a concentration equal to greater than 70% weight of acid/weight of solution, the acid being selected from the group consisting of sulfuric acid and R₅—SO₃H wherein R₅ is C₁-C₄ alkyl optionally substituted with one or more halogen, or R₅ is C₆-C₁₀ aryl optionally substituted with one or more C₁-C₄ alkyl or halogen, to form a compound of formula 3 or a salt of the compound of formula 3.

The methods disclosed herein can form the desired product while reducing the amount of at least one impurity present in the final product, such as epimer formation, the presence of starting reagents, and oxidation by-products. Such reduction in impurities can be achieved during at least one stage of the synthesis, i.e., during any one of the nitration, reduction, and acylation reactions. The methods disclosed herein can also facilitate large-scale synthesis with suitable purities of the final products.

### BRIEF DESCRIPTION OF THE FIGURE

FIG. 1 shows a comparison of the HPLC of the product of the nitration of minocycline with (a) isopropyl nitrate and sulfuric acid and (b) nitric acid and sulfuric acid.

### DETAILED DESCRIPTION OF THE INVENTION

### Definitions

It should be noted that, as used in this specification and the appended claims, the singular forms “a,” “an,” and the include plural referents unless the content clearly dictates otherwise. Thus, for example, reference to a composition containing “a compound” includes a mixture of two or more compounds. It should also be noted that the term or is generally employed in its sense including “and/or” unless the content clearly dictates otherwise.

“Tigecycline” as used herein includes tigecycline in free base form and salt forms, such as any pharmaceutically acceptable salt, enantiomers, and epimers. Tigecycline, as used herein, may be formulated according to methods known in the art. Similarly, “minocycline” as used herein includes minocycline in free base form and salt forms, such as any pharmaceutically acceptable salt, enantiomers, and epimers.

“Compound” as used herein refers to a neutral compound (e.g. a free base), and salt forms thereof (such as pharmaceutically acceptable salts). The compound can exist in anhydrous form, or as a hydrate, or as a solvate. The compound may be present as stereoisomers (e.g., enantiomers and diastereomers), and can be isolated as enantiomers, racemic mixtures, diastereomers, and mixtures thereof. The compound in solid form can exist in various crystalline and amorphous forms.

“Pharmaceutically acceptable” as used herein to refer to those compounds, materials, compositions, and/or dosage forms which are, within the scope of sound medical judgment, suitable for use in contact with the tissues of patients without excessive toxicity, irritation, allergic response, or other problem or complication commensurate with a reasonable risk/benefit ratio.

The term “halogen” as used herein refers to fluoro, chloro, bromo and/or iodo.

The term “alkyl” as used herein refers to a straight- or branched-chain saturated aliphatic hydrocarbon group having from 1-12 carbon atoms, e.g. 1-8, 1-6, or 1-4 carbon atoms.

Examples of alkyl groups include methyl, ethyl, propyl, isopropyl, butyl, isobutyl, sec-butyl, tert-butyl, pentyl, isopentyl, neopentyl, hexyl, heptyl, octyl and the like.

The term “alkylene” as used herein refers to a diradical of a straight or branched-chain saturated aliphatic hydrocarbon group having from 1-6 carbon atoms, e.g. 1-4, 1-3, or 1-2 carbon atoms.

Examples of alkylene groups include methylene, ethylene, trimethylene (1,3-propanediyl), propylene (1,2-propanediyl), tetramethylene (1,4-butanediyl), butylene (1,2-butanediyl), 1,3-butanediyl, 2-methyl-1,3-propanediyl, pentamethylene (1,5-pentanediyl), pentylene (1,2-pentanediyl), hexamethylene (1,6-hexanediyl), hexylene (1,2-hexanediyl), 2,3-dimethyl-1,4-butanediyl and the like.

The term “hydroxy” or “hydroxyl” as used herein refers to an —OH group.

The term “nitro” as used herein refers to the group —NO₂.

The term “amino” as used herein refers to an —NH₂ group.

The term “aryl” as used herein refers to an aromatic hydrocarbon group containing 6-14 carbon ring atoms. “C₆-C₁₄ aryl” refers to a phenyl, naphthyl, biphenyl, anthryl, tetrahydro-naphthyl, fluorenyl, indanyl, biphenylenyl, and acenaphthenyl, groups. Examples of an C₆-C₁₄aryl group include, but are not limited to, phenyl, 1-naphthyl, 2-naphthyl, and 3-biphen-1-yl.

The term “cycloalkyl” as used herein refers to a monocyclic, non-aromatic, saturated hydrocarbon ring containing 3-8 carbon atoms. Representative examples of a C₃-C₈cycloalkyl include, but are not limited to, cyclopropyl, cyclobutyl, cyclopentyl, cyclohexyl, cycloheptyl and cyclooctyl. Each of any two hydrogen atoms on the same carbon atom of the carbocyclic ring can be replaced by an oxygen atom to form an oxo (=O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

Unless indicated otherwise, the nomenclature of substituents that are not explicitly defined herein are arrived at by naming the terminal portion of the functionality followed by the adjacent functionality toward the point of attachment. For example, the substituent “arylalkyloxycabonyl” refers to the group (aryl)-(alkyl)-O-C(O)—.

Each compound in the reaction sequence may be in the form of a free base or of a salt. “Salts” as used herein may be prepared in situ or separately by reacting a free base with a suitable acid. Exemplary salts include, but are not limited to, hydrochloride, hydrobromide, hydroiodide, phosphoric, nitric, sulfuric, acetic, benzoic, citric, cystein, fumaric, glycolic, maleic, succinic, tartaric, sulfate, and chlorobenzensulfonate salts. In another embodiment, the salt can be chosen from alkylsulfonic and arylsulfonic salts. In one embodiment, the compound of formula 2 is provided as a salt, which may be any of the salts disclosed above, such as a hydrochloride salt, or as a sulfate salt.

An “intermediate” as used herein refers to a compound that is formed as an intermediate product between the starting material and the final product. In one embodiment, the intermediate is a product of the nitration of the compound of formula 2. For example, the intermediate can be a compound of formula 3 or a salt thereof.

The intermediate can exist as a free base or as a salt, such as any of the salts disclosed herein. In one embodiment, the intermediate is a sulfate salt.

In one embodiment, an intermediate is not isolated from the reaction mixture. “Reaction mixture” as used herein refers to a solution or slurry comprising a product of a chemical reaction between reagents, as well as by-products, e.g., impurities (including compounds with undesired stereochemistries), solvents, and any remaining reagents, such as starting materials. In one embodiment, the intermediate is the compound of formula 3 and is present in the reaction mixture, which can also contain starting reagents (such as the nitrating agent and/or a compound of formula 2), by-products (such as the C₄-epimer of either formula 2 or formula 3). In one embodiment, the reaction mixture is a slurry, where a slurry can be a composition comprising at least one solid and at least one liquid (such as water, acid, or a solvent), e.g., a suspension or a dispersion of solids.

In another embodiment, an intermediate is isolated from the reaction mixture. In one embodiment, the isolated intermediate is the compound of formula 3.

In one embodiment of the method for the preparation of a compound of formula 3, the method further comprises reacting the compound of formula 3 with a reducing agent to form a compound of formula 4; and (c) optionally forming a salt of the compound of formula 4. The method may further comprise acylating the compound of formula 4 to form a compound of formula 1, and optionally forming a pharmaceutically acceptable salt of the compound of formula 1.

In one embodiment of the method for the preparation of a compound of formula 3, the compound is formed in a reaction mixture and the method further comprises contacting the reaction mixture comprising the compound of formula 3 with a reducing agent to form a compound of formula 4; and (c) optionally forming a pharmaceutically acceptable salt of the compound of formula 4. The method further may comprise acylating the compound of formula 4 to form a compound of formula 1, and optionally forming a pharmaceutically acceptable salt of the compound of formula 1.

In one embodiment, the C₁-C₁₂ alkyl nitrate in any process of the invention is a C₁-C₈ alkyl nitrate, such as a C₃-C₆ alkyl nitrate. The C₁-C₈ alkyl nitrate may be, for example, 2-ethylhexyl nitrate. The C₃-C₆ alkyl nitrate may be, for example, isopropyl nitrate.

In one embodiment, the compounds of formula 3, 4 and 1 are prepared as shown in Scheme 1. Reaction of the compound of formula 2 with a C₁-C₁₂ alkyl nitrate results in insertion of a —NO₂ substituent to form the compound of formula 3. The —NO₂ substituent in formula 3 can be subsequently reduced to an amino, such as by hydrogenation, to form the compound of formula 4. Finally, acylation of the compound of formula 4 generates the compound of formula 1. In Scheme 1, R, R₁, R₂ and n are as further defined herein. Optionally, Scheme 1 may comprise formation of a salt of any one or more of compounds of formula 2, 3, 4 and 1, and the nitration, reduction, and acylation of the respective salts of compounds of formula 2, 3, and 4.

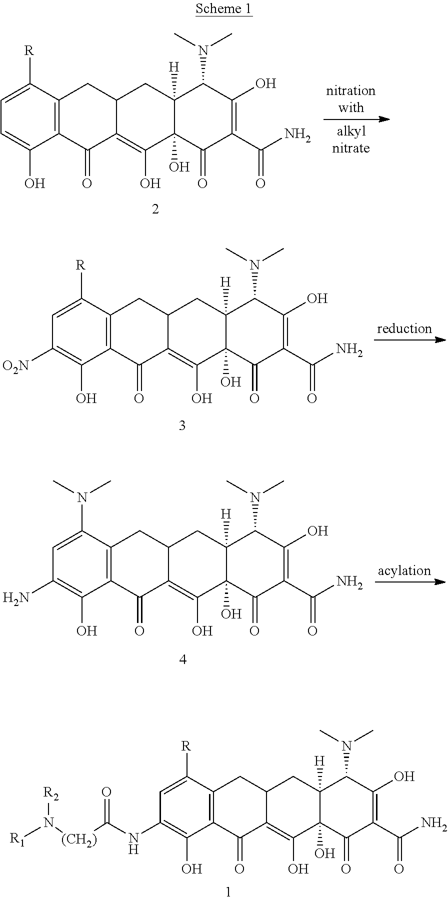

One embodiment discloses a method of preparing a compound of formula 1, or a pharmaceutically acceptable salt thereof, wherein R₁ and R₂ are each independently chosen from hydrogen, (C₁-C₆)alkyl, and cycloalkyl,; R is −NR₃R₄, where R₃ and R₄ are each independently chosen from hydrogen, and (C₁-C₄)alkyl; and n ranges from 1-4.

### Nitration

One embodiment discloses a nitration reaction where the compound of formula 3 is not isolated. Another embodiment discloses a nitration reaction where the compound of formula 3 is isolated.

The C₁-C₁₂ alkyl nitrate can react with the compound of formula 2 or a salt thereof in any solvent deemed suitable by one of ordinary skill in the art. In one embodiment, the acid used is sulfuric acid at a concentration of greater than 70%. Reaction at a concentration of sulfuric acid of 70% was found to be slower than at higher concentrations. Accordingly, the concentration is greater than 70%, such as at least 75%, at least 80%, at least 85%, at least 90%, or at least 95%. For example, the concentration may be 98% or greater.

In one embodiment, the C₁-C₁₂ alkyl nitrate is provided in a molar excess relative to the compound of formula 2. Suitable molar excesses can be determined by one of ordinary skill in the art and can include, but are not limited to, values such as at least 1.05, e.g., a molar excess ranging from 1.05 to 2.5 equivalents, such as a molar excess ranging from 1.05 to 2.0, 1.05 to 1.75, 1.05 to 1.5, or from 1.05 to 1.25, or from 1.05 to 1.1 equivalents.

In one embodiment, the C₁-C₁₂ alkyl nitrate is reacted with the compound of formula 2 by adding the C₁-C₁₂ alkyl nitrate over a period of time. One of ordinary skill in the art can determine a time period over which the total amount of C₁-C₁₂ alkyl nitrate is added to optimize the reaction conditions. For example, the addition of C₁-C₁₂ alkyl nitrate can be monitored by, for example, HPLC, to control the amount of the C₁-C₁₂ alkyl nitrate used. In one embodiment, the total amount of the C₁-C₁₂ alkyl nitrate is added over a period of time of at least 1 h, such as a period of time of at least 2 h, at least 3 h, at least 5 h, at least 10 h, at least 24 h, or a period of time ranging from 1 h to 1 week, ranging from 1 h to 48 h, ranging from 1 h to 24 h, or ranging from 1 h to 12 h.

In one embodiment, the C₁-C₁₂ alkyl nitrate may be added continuously.

In one embodiment, the C₁-C₁₂ alkyl nitrate can be reacted with the compound of formula 2 at a temperature ranging from 0 to 65° C., such as a temperature ranging from 10° C. to 30° C.

It has been surprisingly and unexpectedly found that the use of an alkyl nitrate in the nitration of the compound of formula 1 is advantageous over the use of other nitrating agents. As is further discussed below, advantages include a higher yield using milder conditions that are typically required with other nitrating agents, as well as a better appearance of the product relative to the product obtained from the reaction with other nitrating agents. For example, it is known that the mixture of sulfuric and nitric acids which is commonly used as a nitrating agent is strongly corrosive and its use requires several safety precautions. Additional advantages of the use of an alkyl nitrate in the nitration include: (a) certain alkyl nitrates such as isopropyl nitrate are commercially available; (b) there are no safety issues in the storage and transportation of certain alkyl nitrates such as isopropyl nitrate; and (c) unlike nitric acid, certain alkyl nitrates such as isopropyl nitrate do not have the drawback of reacting with certain acids such as hydrochloric acid (to produce chlorine gas) and therefore may be used with certain acids such as hydrochloric acid.

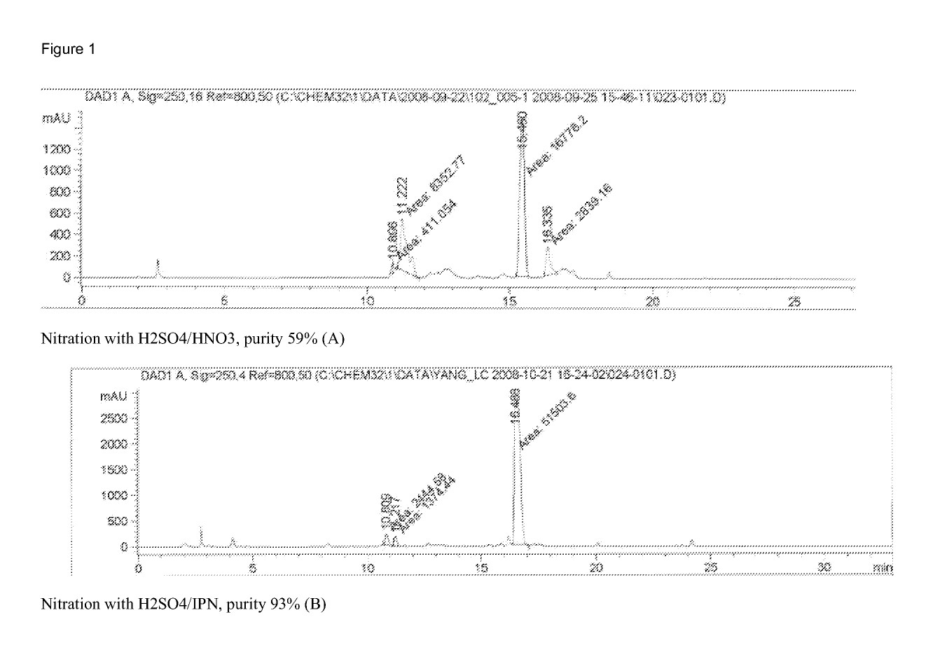

FIG. 1 shows a comparison of the HPLC of the product of the nitration of minocycline with (a) isopropyl nitrate and sulfuric acid and (b) nitric acid and sulfuric acid. It is readily seen that the product in (a) is cleaner, as shown by the smaller number of peaks other than the product peak, and by the smaller size of each of the peaks other than the product peak, in the (a) HPLC plot relative to the (b) HPLC plot. The reaction mixture in (a) has over 93% purity by HPLC, compared with 59% purity in (b).

As an example of a process of the invention, 9-aminominocycline sulfate may be obtained by nitration of minocycline with isopropyl nitrate and sulfuric acid to give 9-nitrominocycline followed by reduction with Pd/C in methanol (Scheme 2). The overall yield is of 87% without isolation of 9-nitrominocycline and 76% with isolation of 9-nitrominocycline. For comparison, the overall yield of the conversion of minocycline to 9-aminominocycline is only 50% when nitration of minocycline is performed with nitric acid and sulfuric acid instead of isopropyl nitrate and sulfuric acid.

In one embodiment, the nitration reaction produces the intermediate of formula 3 while generating an amount of the corresponding C₄-epimer that is less than or equal to 2.5% of the compound of formula 3 as determined by high performance liquid chromatography (HPLC).

HPLC parameters for each step, i.e., nitration, reduction, and acylation, are provided in the Examples section.

In one embodiment, the nitration is performed such that the amount of starting material, e.g., the compound of formula 2, is low. In one embodiment, the compound of formula 2 is present in the nitration product in an amount less than 10%, as determined by HPLC, or less than 5%, less than 3%, less than 2%, less than 1%, or less than 0.5%.

In one embodiment, the nitration can be performed in a large scale. In one embodiment, “large scale” refers to the use of at least 1 gram of the compound according to formula 2, such as the use of at least 2 grams, at least 5 grams, or at least 10 grams.

In one embodiment, R₁ is hydrogen, R₂ is t-butyl, R is —NR₃R₄, where R₃ and R₄ are each methyl, and n is 1. In another embodiment, R₁ and R₂ together with the N they are attached to form a pyrrolidinyl group, R₃ and R₄ are each methyl, and n is 1

In one embodiment, the compound of formula 1 is tigecycline, which may be in the free base form or as the hydrochloride. In this embodiment, the compound of formula 3 is 9-nitro minocycline, which may be in the free base form or as the hydrochloride.

### Reduction

In one embodiment, the process of the invention further comprises reducing the intermediate formed in the nitration step. The reduced intermediate may be a compound of formula 4, or a salt thereof.

The reduction may be performed using a reducing agent such as hydrogen or any other chemical agent that adds hydrogen to a compound. For example, the reduction may be performed under a hydrogen atmosphere at a suitable pressure as determined by one of ordinary skill in the art. In one embodiment, the hydrogen is provided at a pressure ranging from 14 to 100 psi, such as a pressure ranging from 14 to 45 psi., such as, for example, 45 psi.

In another embodiment, the reduction is performed in the presence of at least one catalyst. Exemplary catalysts include, but are not limited to, rare earth metal oxides, Group VIII metal-containing catalysts, and salts of Group VIII metal-containing catalyst. An example of a Group VIII metal-containing catalyst is palladium, such as palladium on carbon.

Where the catalyst is palladium on carbon, in one embodiment, the catalyst is present in an amount ranging from 0.1 parts to 1 part, relative to the amount of the compound of formula 2 present prior to the reaction with the C₁-C₁₂ alkyl nitrate.

In one embodiment, the intermediate is a compound of formula 3. In one embodiment, in the compound of formula 3, R₁ is hydrogen, R₂ is t-butyl, R₃ is methyl, R₄ is methyl, and n is 1.

One of ordinary skill in the art can determine a suitable solvent for the reduction reaction. In one embodiment, prior to the reduction, the reaction mixture is combined with a solvent comprising at least one (C₁-C₈) alcohol and/or at least one (C₁-C₈) diol to form a second reaction mixture. The at least one (C₁-C₈) alcohol can be chosen, for example, from methanol and ethanol. The at least one (C₁-C₈) diol can be, for example, ethylene glycol.

One of ordinary skill in the art can determine a suitable temperature for the reduction reaction. In one embodiment, the reduction is performed at a temperature ranging from 0° C. to 100° C., such as a temperature ranging from 20° C. to 80° C., or from 25 ° C. to 50° C., or from 26° C. to 28° C.

In one embodiment, after the reduction, the resulting reaction mixture is added to or combined with a solvent system comprising a solvent selected from the group consisting of an ether; a halogenated hydrocarbon; a (C₁-C₈) branched chain alcohol; a (C₁-C₈) hydrocarbon; or a combination thereof. In one embodiment, the ether is MTBE, THF, or dibutyl ether. In one embodiment, the halogenated hydrocarbon is DCM or DCE. In one embodiment, the (C₁-C₈) branched chain alcohol is isopropanol. In one embodiment, the (C₁-C₈) hydrocarbon is hexane, heptane, or octane.

In one embodiment, after the reduction, the resulting reaction mixture is added to the solvent system at a temperature ranging from −10° C. to 50° C., such as a temperature ranging from 0° C. to 10° C.

In one embodiment, the method further comprises isolating the compound of formula 4 as a solid, or as a solid composition. In one embodiment, the compound of formula 4 is precipitated or isolated as a salt, such as any of the salts described herein.

In one embodiment, the solid composition comprises a C₄-epimer of formula 4 in an amount less than 10% as determined by high performance liquid chromatography. In another embodiment, the C₄-epimer is present in an amount less than 5%, less than 3%, less than 2%, less than 1%, or less than 0.5%.

In one embodiment, the solid composition comprises the formula 2 in an amount less than 2%, such as an amount less than 1%, or less than 0.5%, as determined by high performance liquid chromatography.

In one embodiment, the reduction can be performed in a large scale. In one embodiment, “large scale” refers to the use of at least 1 gram of the compound according to formula 2, such as the use of at least 2 grams, at least 5 grams, at least 10 grams, at least 25 gram, at least 50 grams, at least 100 grams, at least 500 g, at least 1 kg, at least 5 kg, at least 10 kg, at least 25 kg, at least 50 kg, or at least 100 kg.

In one embodiment, the acylation is perormed with an aminoacyl compound as the acylating agent. In one embodiment, the acylation is performed in a reaction medium that may be chosen from an aqueous medium, and at least one basic solvent in the absence of a reagent base.

In one embodiment, the method for preparing a compound of formula I is a method for preparing tigecycline or a pharmaceutically acceptable salt thereof.

The salt of the compound of Formula 4 may be a halogenated salt, such as a hydrochloride salt.

The acylation reaction medium may be a solvent chosen from a polar aprotic solvent or mixture of solvents thereof. In one embodiment, the polar aprotic solvent is chosen from acetonitrile, 1,2-dimethoxyethane, dimethylacetamide, dimethylformamide, hexamethylphosphoramide, N,N′-dimethylethyleneurea, N,N′-dimethylpropyleneurea, methylene chloride, N-methylpyrrolidinone, tetrahydrofuran, and mixtures thereof. In another embodiment, the polar aprotic solvent is chosen from acetonitrile, dimethylformamide, N,N′-dimethylpropyleneurea, N-methylpyrrolidinone, tetrahydrofuran, and mixtures thereof. The at least one basic solvent may be a mixture of acetonitrile and N,N′-dimethylpropyleneurea. In another embodiment, the at least one basic solvent may be a mixture of water and N,N′-dimethylpropyleneurea. In a further embodiment, the at least one basic solvent is N,N′-dimethylpropyleneurea.

The reaction medium may be an aqueous medium. In a further embodiment, the at least one basic solvent in the absence of a base is water in the absence of a base. In another embodiment, the reaction medium may be at least one basic solvent in the absence of a reagent base. A basic solvent is a solvent capable of accepting, either partially or fully, a proton. A reagent base refers to a base that is added at the start of the reaction, either concurrently or sequentially with the compound of Formula 4 and the aminoacyl compound and is capable of accepting, either partially or fully, a proton. A reagent base also refers to a base that is added during the reaction.

The aminoacyl compound may be chosen from aminoacyl halides, aminoacyl anhydrides, and mixed aminoacyl anhydrides. In one embodiment, the aminoacyl compound is an aminoacyl halide of Formula 6:

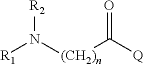

or a salt thereof,

wherein R₁ and R₂ are each independently chosen from hydrogen, (C₁-C₆)alkyl, and cycloalkyl; and wherein Q is a halogen chosen from fluoride, bromide, chloride, and iodide.

In a further embodiment, Q is chloride. The salt of the compound of Formula 6 may be chosen from a halogenated salt. Halogenated salt refers to any salt formed from interaction with a halogen anion, such as a hydrochloride salt, a hydrobromide salt, and a hydroiodic salt. In one embodiment, the halogenated salt is a hydrochloride salt.

In another embodiment, n is one. In a further embodiment, X is bromide.

Reacting a compound of Formula 4 with the aminoacyl compound may be conducted at a temperature ranging from 0° C. to 30° C., such as from 20° C. to 25° C., such as from 10° C. to 17° C., such as from 0° C. to 6° C., and further such as from 2° C. to 8° C. The time period for reaction may range from 1 hour to 24 hours, such as from 0.5 hours to 4 hours, and further such as from 2 hours to 8 hours. An excess of aminoacyl compound relative to the amount of a compound of Formula 4 may be used in the reaction. In one embodiment, the excess may be 3 equivalents of aminoacyl compound to 1 equivalent of the compound of Formula 4. In another embodiment, the ratio of aqueous medium to the compound of Formula 4 may be 6:1 w/w or 5:1 volumes. In one embodiment, the aminoacyl compound is added to or combined with a solution of the compound of Formula 4 in an aqueous medium.

In one embodiment, where the reaction medium is an aqueous medium, the pH of the aqueous medium may be adjusted to a pH ranging from 4 to 9, such as from 5 to 7.5, such as from 6.3 to 6.7, such as from 7.0 to 7.5, further such as 6.5, and still further such as 7.2. Water may be added prior to adjusting the pH. Adjusting the pH may involve addition of a base, including but not limited to ammonium hydroxide. The concentration of ammonium hydroxide may range from 25% to 30%. In another embodiment, an acid, such as hydrochloric acid, may be used to adjust the pH. The reaction medium during pH adjustment may be at a temperature ranging from −5° C. to 8° C., such as from 5° C. to 8° C., and further such as from 0° C. to 5° C.

Following adjustment of the pH, at least one organic solvent or mixture of solvents may be added to the aqueous medium. In one embodiment, the at least one organic mixture of solvents may comprise methanol and methylene chloride. The concentration of methanol may range from 5% to 30%, including but not limited to 20% and 30%. In another embodiment, the at least one organic solvent or mixture of solvents comprises tetrahydrofuran. The temperature of the mixture may range from 15° C. to 25° C.

In one embodiment, the aqueous medium may be extracted with a mixture of at least one polar protic solvent and at least one polar aprotic solvent. In one embodiment, the at least one polar aprotic solvent comprises methylene chloride and the at least one polar protic solvent comprises methanol. In another embodiment, the aqueous medium is extracted with at least one polar aprotic solvent, such as methylene chloride. The extraction may be conducted at a temperature ranging from −5° C. to 25° C., further such as from 0° C. to 5° C. In a further embodiment, the pH of the aqueous medium is adjusted to a range from 7.0 to 7.5, such as 7.2, after each extraction. The extraction process may be repeated, for example, up to 10 times.

In one embodiment, the combined organic extracts may be treated with a drying agent, such as sodium sulfate. The organic extracts may also be treated with charcoal, such as Norit CA-1. The solids are removed by filtration to give a solution. In one embodiment, the solution may be concentrated to afford the compound of Formula 1.

The compound of Formula 1 obtained from the reaction may be crystallized in at least one organic solvent or mixture of solvents. In one embodiment, the organic mixture of solvents comprises methanol and methylene chloride. Crystallization may, for example, occur at a temperature ranging from −15° C. to 155° C., such as from 0° C. to 15° C., and further such as from 2° C. to 5° C.

In another embodiment, following extraction, the resulting organic mixture of at least one polar protic solvent and at least one polar aprotic solvent may be concentrated to give slurry and filtered to give the compound of Formula 1. Concentration and filtration may, for example, occur at 0° C. to 5° C.

A method for preparing a compound of Formula 1 may be performed using greater than 5 grams of the amine of Formula 4, such as greater than 10 grams, such as greater than 50 grams, such as greater than 100 grams, such as greater than 500 grams, such as greater than 1 kilograms, and further such as greater than 10 kilograms.

In one embodiment, the compound of Formula 1 prepared by any of the methods described herein contains less than 10.0% impurities as determined by high performance liquid chromatography, such as less than 5% impurities, such as less than 2% impurities, and further such as 1-1.4% impurities. In a further embodiment, the compound of Formula 1 contains a C₄-epimer in an amount less than 1.0% as determined by high performance liquid chromatography, such as less than 0.5% C₄-epimer, and further such as less than 0.2% C₄-epimer. In one embodiment, the compound of formula 1 contains less that 1% minocycline as determined by high performance liquid chromatography, such as less than 0.6% minocycline. In another embodiment, the compound of formula 1 contains less than 5% dichloromethane, such as less than 2-3% dichloromethane.

### Purification

In one embodiment of the invention, the process may further comprise purifying a compound of Formula 1:

or a pharmaceutically acceptable salt thereof,

or a pharmaceutically acceptable salt thereof, wherein R₁ and R₂ are each independently chosen from hydrogen, straight and branched chain (C₁-C₆)alkyl, and cycloalkyl; R is —NR₃R₄, where R₃ and R₄ are each independently chosen from hydrogen, and straight and branched chain (C₁-C₄)alkyl; and n ranges from 1-4, the purification comprising:

A) combining the compound of Formula 1 with at least one polar aprotic solvent and at least one polar protic solvent to give a first mixture,

B) mixing the first mixture for at least one period of time such as from 15 minutes to 2 hours at a temperature ranging from 0° C. to 40° C., and

C) obtaining the compound of Formula 1.

As used herein, the term “obtaining” refers to isolating a compound at a useful level of purity, including but not limited to levels of purity greater than 90%, 95%, 96%, 97%, 98%, and 99%. The level of purity may be determined by high pressure liquid chromoatography.

In one embodiment, the purification of a compound of Formula 1 involves the steps of:

A) combining the compound of Formula 1 with at least one polar aprotic solvent and at least one polar protic solvent to give a first mixture,

B) mixing the first mixture for a period of time at a temperature ranging from 30° C. to 40° C.,

C) cooling the first mixture to a temperature ranging from 15° C. to 25° C. and allowing the mixture to stand without mixing for a second period of time,

D) cooling the first mixture to a temperature ranging from 0° C. to 6° C. and allowing the mixture to stand without mixing for a third period of time, and

E) obtaining the compound of Formula 1.

In one embodiment, the method may include a compound of Formula 1 where n is 1, R₁ is hydrogen, R₂ is t-butyl, and R₃ and R₄ are each methyl.. The compound of Formula 1 that is combined with the at least one polar aprotic solvent and the at least one polar protic solvent may be provided in a form chosen from a solid, a slurry, a suspension, and a solution.

In one embodiment, the at least one polar aprotic solvent may chosen from acetone, 1,2-dichloroethane, methyl acetate, methyl ethyl ketone, methyl isobutyl ketone, methylene chloride, and ethyl acetate. In a further embodiment, the at least one polar aprotic solvent may be chosen from acetone and methylene chloride. In another embodiment, the at least one polar protic solvent may be chosen from methanol, ethanol, isopropanol, and t-butanol. In a further embodiment, the at least one polar protic solvent may be methanol.

The combination of the at least one polar aprotic solvent and at least one polar protic solvent may include acetone and methanol. Another embodiment provides a combination of the at least one polar aprotic solvent, methylene chloride, and the at least one polar protic solvent, methanol. In a further embodiment, the combination of the at least one polar aprotic solvent and at least one polar protic solvent may include methyl acetate and methanol. The compound of Formula 1 may, for example, be combined with equal volumes of the at least one polar aprotic solvent and the at least one polar protic solvent.

In one embodiment, the first mixture may, for example, be mixed for a first period of time ranging from 30 minutes to 2 hours where the temperature ranges from 15° C. to 25° C., then for a second period of time ranging from 30 minutes to 2 hours, where the temperature ranges from 0° C. to 2° C. In one embodiment, the first period of time and the second period of time are each 1-hour. In another embodiment, the method may comprise mixing the first mixture for at least one period of time ranging from 30 minutes to 2 hours at a temperature ranging from 15° C. to 25° C., then filtering the first mixture to obtain a solid. The method may further comprise combining the solid with at least one polar aprotic solvent and at least one polar protic solvent, such as at equal volumes, for a first period of time ranging from 30 minutes to 2 hours at a temperature ranging from 15° C. to 25° C., and filtering to obtain a second solid. In a further embodiment, these combining and filtering steps may be repeated two to fifteen times.

Purification of a compound of Formula 1 may further comprise obtaining a solid from the first mixture, and combining the solid with at least one polar protic solvent and at least one polar aprotic solvent to obtain a second mixture. The second mixture may, for example, comprise methanol and methylene chloride in a ratio by volume ranging from 1:5 to 1:15 methanol:methylene chloride. In one embodiment, the second mixture may be mixed at a temperature ranging from 30° C. to 36° C. and then filtered to obtain a solution. In a further embodiment, the concentration of the polar protic solvent in the solution may be reduced to a level below 5%, and the solution may be mixed, for example, at a temperature ranging from 0° C. to 6° C., for a time period, for example, ranging from 30 minutes to 2 hours prior to filtering.

In one embodiment, mixing the first mixture may occur during a period of time ranging from 10 to 20 minutes, such as 15 minutes. In one embodiment, cooling the first mixture to a temperature ranging from 15° C. to 25° C. and allowing the mixture to stand without mixing may occur during a second period of time ranging from 30 minutes to 3 hours, such as from 1 hour to 2 hours. The first mixture may be further cooled to a temperature ranging from 0° C. to 6° C. and allowed to stand without mixing for a third period of time ranging from 30 minutes to 2 hours, such as 1 hour.

Obtaining the compound of Formula 1 may include filtering any mixture described herein through at least one filter selected from pyrogen reducing filters and clarifying filters. As disclosed herein, mixing may be carried out by using a mechanical mixing device, for instance, a stirrer or agitator. Mixing may also be effected by solubility of the compound having Formula 1 in the solvent system. Increasing the temperature may increase solubility.

In one embodiment, when a compound of Formula 1 is to be combined with at least one polar aprotic solvent and at least one polar protic solvent, the compound of Formula 1 may be used in the form of a pharmaceutically acceptable salt thereof. Where a compound of Formula 1 is obtained as the product of the method of the invention, the compound of Formula 1 may be recovered in the form of a pharmaceutically acceptable salt thereof.

In another embodiment, where a compound of Formula 1 is obtained by the method according to the invention, the compound may be converted into a pharmaceutically acceptable salt thereof by addition of an acid.

In one embodiment, the purified compound of formula 1 is tigecycline.

The tigecycline that is combined with at least one polar aprotic solvent and at least one polar protic solvent may be provided in a form chosen from a solid, a slurry, a suspension, and a solution. In one embodiment, the tigecycline obtained from the method may contain less than 1% of the C-4 epimer of tigecycline or a pharmaceutically acceptable salt thereof as determine by high-pressure liquid chromatography (HPLC).

The compound of Formula 1 obtained from the method may contain less than 3.0% impurities as determined by HPLC, such as less than 1.0% impurities, such as less than 0.7% impurities. In another embodiment, the compound of Formula 1 may contain less than 2% of the C₄ epimer of the compound of formula 1 or a pharmaceutically acceptable salt thereof, as determined by HPLC, such as less than 1% of the C₄ epimer, such as less than 0.5% of the C₄ epimer.

The method may be performed on greater than 5 grams of the compound of Formula 1, such as greater than 50 grams, such as greater than 100 grams, such as greater than 500 grams, such as greater than 1 kilogram, and further such as greater than 10 kilograms.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The following examples are intended to illustrate the invention in a non-limiting manner.

### EXAMPLES

HPLC analyses were performed under the following conditions:
Column: Waters Symmetratry RP8 15×0.46 cm
Mobile phase: A; 0.03M KH2PO4, pH 2 with H3PO4, B; 9:1 acetonitrile:water
Flow rate: 0.8 ml/min
Detection wavelenghth: 250 nm
Column oven temp. 35C
Isocratic program:

| Time (min) | A (%) | B (%) |
|---|---|---|
| 0 | 90 | 10 |
| 2 | 90 | 10 |
| 30 | 45 | 55 |
| 32 | 90 | 10 |

### Nitration

Minocycline, an example of a compound of formula 2, was prepared according to the method described in U.S. Pat. No. 3,226,436. Minocycline chloride was then nitrated according to the following procedure:

To 15 g of 98% sulfuric acid was added 5.3 g of minocycline chloride in several portions at 10-30° C. The suspension was stirred under N₂ for 4 hrs at 20° C. forming a homogeneous solution. To this solution was added 2.2 mL of isopropyl nitrate slowly to maintain the reaction temperature below 30° C. After addition the reaction was aged for another 2 hrs. The reaction mixture was slowly added to IPA/Hep solution (125 mL/25 mL) below 10° C. and then the solution was agitated for another 1 hr at this temperature. The formed solid was filtered and washed with pre-cooled 30 mL of IPA. The solid was dried at vacuum oven at 40° C. for 20 hrs. A yellow solid (7.3 g, 91.3% wt purity, 92.6% yield) was obtained for next reaction without further purification.

Reduction after isolation of an intermediate formed from the nitration reaction

To a pressured glass bottle was added 50 mL of methanol and 10 mL of sulfuric acid. Previously prepared 9-nitrominocycline sulfate 5 g was added at 5° C. in one potion. To the solution was added 0.4g of 10% Pd/C and then the suspension was purged with N₂ for three times and with H₂ once. The suspension was maintained under H₂ at a pressure of 45 PSI for 3 hr at 20° C. in a Parr Shaker. After removing the catalyst by a simple filtration, the reaction solution was added to IPA/Hep solution (100 mL/30 mL) at 5-10° C. The solid was filtered and washed with pre-cooled 20 mL of IPA. The solid was dried at 40° C. for 20 hrs in a vacuum oven affording a yellow solid (4.3 g, 74% wt pure) with a yield of 76%.

Reduction without isolation of any intermediate formed from the nitration reaction

To 15 g of 98% sulfuric acid was added 5.3 g of minocycline chloride in several portions at 10-30° C. The suspension was stirred under N₂ for 4 hrs at 20° C. forming a homogeneous solution. To this solution was added 2.2 mL of isopropyl nitrate slowly to maintain the reaction temperature below 30° C. After addition the reaction was aged for another 2 hrs. The reaction mixture was diluted with 50 mL of methanol at 5-10° C. and then added 1 g of 10% Pd/C. The suspension was purged with N₂ for three times, with H₂ once and then maintained under H₂ at a pressure of 45 PSI for 3 hrs in a Parr shaker. After removing the catalyst by a simple filtration, the reaction solution was added to IPA/Hep solution (250 mL/50 mL) at 5 -10° C. The solid was filtered and washed with pre-cooled 40 mL of IPA. The solid was dried at 40° C. for 20 hrs in a vacuum oven affording a yellow solid (7.4g, 72% wt pure) with a yield of 87%.

### Acylation

The acylation of the compound of formula 4 may be performed as disclosed above. The Acylation is also disclosed in U.S. Patent Publication No. 2007-0049560 A1 paragraphs [0143]-[0213], incorporated by reference herein.

### Purification

The Purification of the compound of formula 1 may be performed as disclosed above. Purification is also disclosed in U.S. Patent Publication No. 2007-0049560 A1 paragraphs [0214]-[0283], incorporated by reference herein.

While the invention has been described by discussion of embodiments of the invention and non-limiting examples thereof, one of ordinary skill in the art may, upon reading the specification and claims, envision other embodiments and variations which are also within the intended scope of the invention and therefore the scope of the invention shall only be construed and defined by the scope of the appended claims.

What is claimed is:

1. A method of preparing a compound of formula 1, 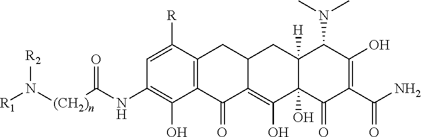
or a pharmaceutically acceptable salt thereof, wherein R₁ and R₂ are each independently chosen from hydrogen, (C₁-C₆)alkyl, and cycloalkyl, R is —NR₃R₄, where R₃ and R₄ are each independently chosen from hydrogen, and (C₁-C₄)alkyl; and n ranges from 1-4, comprising:
(a) reacting a C₁-C₁₂ alkyl nitrate with a compound of formula 2, 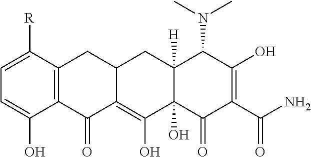
or a salt thereof, in the presence of an acid at a concentration greater than 70% weight of acid/weight of solution, the acid being selected from the group consisting of sulfuric acid and R₅—SO₃H wherein R₅ is C₁-C₄ alkyl optionally substituted with one or more halogen, or R₅ is C₆-C₁₀ aryl optionally substituted with one or more C₁-C₄ alkyl or halogen, to produce a reaction mixture containing a compound of formula 3 or a salt thereof 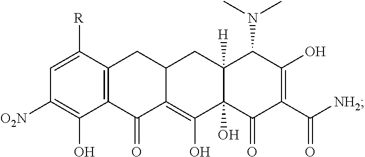
(b) reducing the compound of formula 3 or a salt thereof to form a compound of formula 4 or a salt thereof 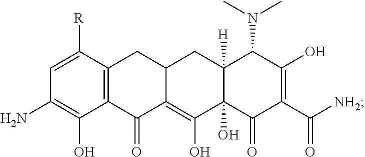
(c) acylating the compound of formula 4 to form a compound of formula 1; and
(d) optionally forming a pharmaceutically acceptable salt of the compound of formula 1.

2. The method of claim 1, wherein the compound of formula 3 is isolated from the reaction mixture.

3. The method of claim 1, compound of formula 3 is not isolated from the reaction mixture prior to step (b).

4. The method of claim 1, wherein the acid is sulfuric acid

5. The method of claim 1, wherein the sulfuric acid has a concentration of at least 95%.

6. The method of claim 1, wherein the alkyl nitrate is present in a molar excess relative to the compound of formula 2.

7. The method of claim 6, wherein the molar excess is at least 1.05 equivalents.

8. The method of claim 1, wherein the reacting in (a) is at a temperature ranging from 10° C. to 30° C.

9. The method of claim 1, wherein the reacting in (a) is with a salt of a compound of formula 2.

10. The method of claim 9, wherein the salt of the compound of formula 2 is selected from the group consisting of hydrochloride, hydrobromide, hydroiodide, phosphoric, nitric, sulfuric, acetic, benzoic, citric, cystein, fumaric, glycolic, maleic, succinic, tartaric, sulfate, and chlorobenzensulfonate salts.

11. The method of claim 9, wherein the salt of the compound of formula 2 is a hydrochloride or a sulfate.

12. The method of claim 1, wherein the reaction mixture includes the C₄-epimer of formula 3 in an amount less than or equal to 2.5% of the compound of formula 3, as determined by high performance liquid chromatography.

13. The method of claim 1, wherein R₁ is hydrogen, R₂ is t-butyl, R₃ is methyl, R₄ is methyl, and n is 1.

14. The method of claim 1, wherein the compound of formula 1 is tigecycline.

15. The method of claim 1, wherein the compound of formula 1 is tigecycline.HCl.

16. The method of claim 1, wherein the C₁-C₁₂ alkyl nitrate is a C₁-C₈ alkyl nitrate.

17. The method of claim 16, wherein the C₁-C₈ alkyl nitrate is 2-ethylhexyl nitrate.

18. The method of claim 16, wherein the C₁-C₈ alkyl nitrate is a C₃-C₆ alkyl nitrate.

19. The method of claim 16, wherein the C₃-C₆ alkyl nitrate is isopropyl nitrate.

20. A method of preparing a compound of formula 3, 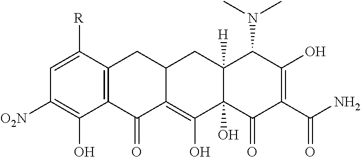
or a salt thereof, wherein R₁ and R₂ are each independently chosen from hydrogen, (C₁-C₆)alkyl, and cycloalkyl,; R is —NR₃R₄, where R₃ and R₄ are each independently chosen from hydrogen, and (C₁-C₄)alkyl; and n ranges from 1-4, comprising reacting a C₁-C₁₂ alkyl nitrate with a compound of formula 2, 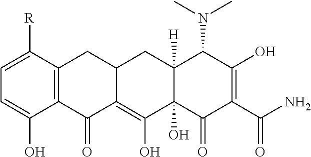
or a salt thereof, in the presence of an acid at a concentration greater than 70% weight of acid/weight of solution, the acid being selected from the group consisting of sulfuric acid and R₅—SO₃H wherein R₅ is C₁-C₄ alkyl optionally substituted with one or more halogen, or R₅ is C₆-C₁₀ aryl optionally substituted with one or more C₁-C₄ alkyl or halogen, to form a compound of formula 3 or a salt of the compound of formula 3.

21. The method of claim 20, wherein the compound of formula 3 is 9-nitro minocycline, or a pharmaceutically acceptable salt thereof.

22. The method according to claim 20, wherein the compound of formula 3 is 9-nitro minocycline.HCl.

23. The method of claim 20, wherein the C₁-C₁₂ alkyl nitrate is a C₁-C₈ alkyl nitrate.

24. The method of claim 23, wherein the C₁-C₈ alkyl nitrate is 2-ethylhexyl nitrate.

25. The method of claim 23, wherein the C₁-C₈ alkyl nitrate is a C₃-C₆ alkyl nitrate.

26. The method of claim 23, wherein the C₃-C₆ alkyl nitrate is isopropyl nitrate.
